(19) United States
(12) Patent Application Publication
(10) Pub. No.: US 2021/0386889 A1
(43) Pub. Date: Dec. 16, 2021

(54) AUTOMATED DOOR HANDLE / PANEL SANITIZER DISPENSING ASSEMBLY

(71) Applicant: Board of Supervisors of Louisiana State University and Agricultural and Mechanical College, Baton Rouge, LA (US)

(72) Inventors: Giovanni Francesco SOLITRO, Shreveport, LA (US); Johnathan Steven ALEXANDER, Shreveport, LA (US); R. Shane BARTON, Shreveport, LA (US)

(73) Assignee: Board of Supervisors of Louisiana State University and Agricultural and Mechanical College, Baton Rouge, LA (US)

(21) Appl. No.: 17/386,521

(22) Filed: Jul. 27, 2021

Related U.S. Application Data
(60) Provisional application No. 63/030,347, filed on May 27, 2020.

(51) Int. Cl. A61L 2/18; A61L 2/10

(52) U.S. Cl. CPC A61L 2/18; E05B 1/0069; A61L 2/10

(57) ABSTRACT

An automatic door handle/panel sanitizer dispensing assembly comprising a frame, a control unit including a processor, an actuator assembly controlled by the processor, the actuator assembly includes one of a pump and a valve, a first sensor operatively connected to the processor, a power storage unit powering the control unit, the actuator assembly, and the first sensor, a first nozzle and a second nozzle, both fluidly connected to the actuator assembly, a container coupled to the frame, for storing disinfectant, a threaded hook to hang the assembly onto the door handle, a tray having an arcuate shape and being arranged abutting a roof of the frame and being supported by the frame, a plurality of threaded tray holes extending through the tray arranged proximate to a rear end of the tray.

### CROSS REFERENCE TO RELATED APPLICATIONS/PRIORITY

The present invention claims priority to U.S. Provisional Patent App. Ser. No. 63/030,347 filed May 27, 2020, which is incorporated by reference into the present disclosure as if fully restated herein. Any conflict between the incorporated material and the specific teachings of this disclosure shall be resolved in favor of the latter. Likewise, any conflict between an art-understood definition of a word or phrase and a definition of the word or phrase as specifically taught in this disclosure shall be resolved in favor of the latter.

### TECHNICAL FIELD

The present disclosure relates, generally, to a sanitizer dispensing assembly. More particularly, the present disclosure relates to a sanitizer dispensing assembly suitable for dispensing a disinfectant on the door handles.

### BACKGROUND

Exposure to viruses and bacteria in entrance and exit doors is a danger to individuals of various age ranges, but especially children and elderly. Various ineffective solutions as disinfecting door handles generally have the disadvantage requiring replacement of the door or door handle, disinfect only a portion of the door handle surface, such that the sanitization of the entire door handle surface is not assured, and cause disinfectant solution to drip on the floor, making the floor wet which can create slippery and dangerous conditions for individuals quickly traversing the area.

### SUMMARY

Wherefore, it is an object of embodiments of the present invention to overcome one or more of the above-mentioned shortcomings and drawbacks associated with the current technology.

The disclosed invention relates to an automatic door handle/panel sanitizer dispensing assembly comprising a frame, a control unit including a processor, an actuator assembly controlled by the processor, a first sensor operatively connected to the processor, a power storage unit powering the control unit, the actuator assembly, and the first sensor, and a first nozzle fluidly connected to the actuator assembly. According to a further embodiment the assembly further comprises a second nozzle fluidly connected to actuator assembly. According to a further embodiment the assembly further comprises a container coupled to the frame, for storing disinfectant. According to a further embodiment the actuator assembly includes one of a pump and a valve. According to a further embodiment the assembly further comprises a tray having an arcuate shape and being arranged abutting a roof of the frame and being supported by the frame. According to a further embodiment the assembly further comprises a plurality of tray holes extending through the tray arranged proximate to a rear end of the tray. According to a further embodiment the assembly further comprises a hook to hang the assembly onto the door handle and wherein each tray hole facilitates an extension of the hook through the tray and inside an aligned frame hole of the frame, and enables an attachment of the hook with the frame. According to a further embodiment the hook is threaded and the frame holes are threaded to enable a threaded engagement of the hook with the frame. According to a further embodiment the assembly further comprises an arch shaped handle removably attached to the tray and adapted to support one of the first nozzle, a second nozzle, and both the first nozzle and the second nozzle. According to a further embodiment the first sensor is one of a proximity sensor, a motion sensor, and a position sensor. According to a further embodiment the control unit is programed such that when the first sensor detects an object is one of in proximity to the door handle and touches the door handle, and then the object is removed from the door handle, the processor causes the actuator assembly to spray disinfectant on the door handle. According to a further embodiment the control unit is programed such that when the first sensor detects the door is opened and then closed again, the processor causes the actuator assembly to spray disinfectant on the door handle. According to a further embodiment the assembly further comprises a UV light source controlled by the control unit and powered by the power storage unit, the UV light source arranged to direct UV light rays onto the door handle when actuated by the control unit. According to a further embodiment the assembly further comprises a heating element and a fan, both controlled by the control unit and powered by the power storage unit, the fan arranged to direct heated air onto the door handle when actuated by the control unit.

The disclosed invention further relates to an automatic door handle/panel sanitizer dispensing assembly comprising a frame, a control unit including a processor, an actuator assembly controlled by the processor, the actuator assembly includes one of a pump and a valve, a first sensor operatively connected to the processor, a power storage unit powering the control unit, the actuator assembly, and the first sensor, a first nozzle and a second nozzle, both fluidly connected to the actuator assembly, a container coupled to the frame, for storing disinfectant, a threaded hook to hang the assembly onto the door handle, a tray having an arcuate shape and being arranged abutting a roof of the frame and being supported by the frame, a plurality of threaded tray holes extending through the tray arranged proximate to a rear end of the tray, wherein each tray hole facilitates an extension of the hook through the tray and inside an aligned frame hole of the frame, and enables an attachment of the hook with the frame, an arch shaped handle removably attached to the tray and adapted to support one of the first nozzle, a second nozzle, and both the first nozzle and the second nozzle, and one of a UV light source controlled by the control unit and powered by the power storage unit, the UV light source arranged to direct UV light rays onto the door handle when actuated by the control unit, and a heating element and a fan, both controlled by the control unit and powered by the power storage unit, the fan arranged to direct heated air onto the door handle when actuated by the control unit, wherein the first sensor is one of a proximity sensor, a motion sensor, and a position sensor, and wherein the control unit is programed such that one of when the first sensor detects an object is one of in proximity to the door handle and touches the door handle, and then the object is removed from the door handle, the processor causes the actuator assembly to spray disinfectant on the door handle, and when the first sensor detects the door is opened and then closed again, the processor causes the actuator assembly to spray disinfectant on the door handle.

Various objects, features, aspects, and advantages of the present invention will become more apparent from the following detailed description of preferred embodiments of the invention, along with the accompanying drawings in which like numerals represent like components. The present invention may address one or more of the problems and deficiencies of the current technology discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

### BRIEF DESCRIPTION OF THE DRAWINGS

The accompanying drawings, which are incorporated in and constitute a part of the specification, illustrate various embodiments of the invention and together with the general description of the invention given above and the detailed description of the drawings given below, serve to explain the principles of the invention. It is to be appreciated that the accompanying drawings are to scale for at least one embodiment, but the emphasis is placed on illustrating the principles of the invention. The invention will now be described, by way of example, with reference to the accompanying drawings in which:

FIG. 1 illustrates a sanitizer dispensing assembly mounted to a door handle of a door, in accordance with an embodiment of the disclosure;

FIG. 2 is perspective view of the sanitizer dispensing assembly of FIG. 1, in accordance with an embodiment of the disclosure;

FIG. 3 is front view of the sanitizer dispensing assembly, in accordance with an embodiment of the disclosure;

FIG. 4 is a rear view of the sanitizer dispensing assembly, in accordance with an embodiment of the disclosure;

FIG. 5 is a an exploded view of the sanitizer dispensing assembly, in accordance with an embodiment of the disclosure

FIG. 6 is a perspective view of a frame of the sanitizer dispensing assembly, in accordance with an embodiment of the disclosure;

FIG. 7 is a front perspective of the sanitizer dispensing assembly with a cover and a first nozzle removed, in accordance with an embodiment of the disclosure;

FIG. 8 is a sectional perspective view of the sanitizer dispensing assembly, in accordance with an embodiment of the disclosure;

FIG. 9 is a sectional front view of the sanitizer dispensing assembly, in accordance with an embodiment of the disclosure;

FIG. 10 is an exploded view of the sanitizer dispensing assembly, in accordance with an alternative embodiment of the disclosure; and

FIG. 11 illustrates a bottle attached to the frame of the sanitizer dispensing assembly using an adapter plate, in accordance with an alternative embodiment of the disclosure.

### DETAILED DESCRIPTION

The present invention will be understood by reference to the following detailed description, which should be read in conjunction with the appended drawings. It is to be appreciated that the following detailed description of various embodiments is by way of example only and is not meant to limit, in any way, the scope of the present invention. In the summary above, in the following detailed description, in the claims below, and in the accompanying drawings, reference is made to particular features (including method steps) of the present invention. It is to be understood that the disclosure of the invention in this specification includes all possible combinations of such particular features, not just those explicitly described. For example, where a particular feature is disclosed in the context of a particular aspect or embodiment of the invention or a particular claim, that feature can also be used, to the extent possible, in combination with and/or in the context of other particular aspects and embodiments of the invention, and in the invention generally. The terms “comprise(s),” “include(s),” “having,” “has,” “can,” “contain(s),” and grammatical equivalents and variants thereof, as used herein, are intended to be open-ended transitional phrases, terms, or words that do not preclude the possibility of additional acts or structures. are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components. The singular forms “a,” “and” and “the” include plural references unless the context clearly dictates otherwise. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

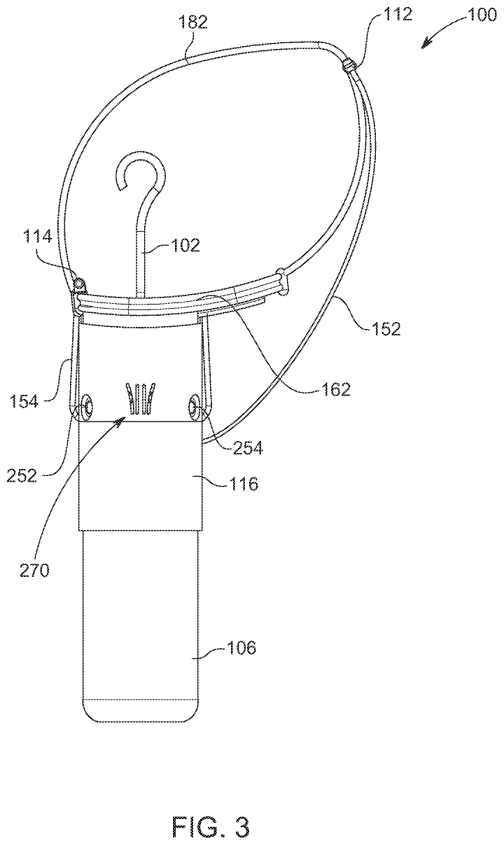

The term “at least” followed by a number is used herein to denote the start of a range beginning with that number (which may be a range having an upper limit or no upper limit, depending on the variable being defined). For example “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40% means 40% or less than 40%. When, in this specification, a range is given as “(a first number) to (a second number)” or “(a first number)-(a second number),” this means a range whose lower limit is the first number and whose upper limit is the second number. For example, 25 to 100 mm means a range whose lower limit is 25 mm, and whose upper limit is 100 mm. Where spatial directions are given, for example above, below, top, and bottom, such directions refer to the sanitizer dispensing assembly as represented in FIG. 3, unless identified otherwise.

The embodiments set forth the below represent the necessary information to enable those skilled in the art to practice the invention and illustrate the best mode of practicing the invention. For the measurements listed, embodiments including measurements plus or minus the measurement times 5%, 10%, 20%, 50% and 75% are also contemplated. For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

In addition, the invention does not require that all the advantageous features and all the advantages of any of the embodiments need to be incorporated into every embodiment of the invention.

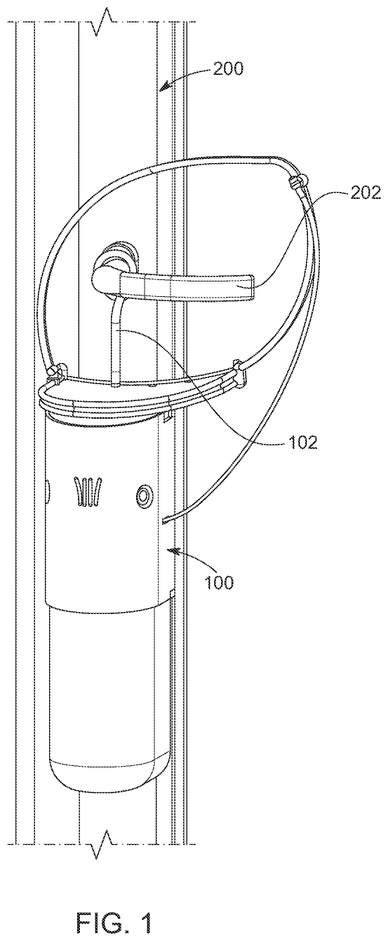

Reference will be made to the figures, showing various embodiments of a sanitizer dispensing assembly. Referring to FIG. 1, a sanitizer dispensing assembly 100 (hereinafter simply referred to as a dispenser 100) mounted on a door handle 202 of a door 200 using a hook 102 is shown. The dispenser 100 is automatically actuated to dispense a disinfectant, for example, a disinfecting fluid or a cleaning fluid, on the door handle 202 and in the vicinity of the door handle 202 to spray the disinfectant on the door handle 202 and preferably not on a person operating the door handle 202. The dispenser 100 is adapted to dispense the disinfectant in response to the actuation of the door handle 202 and/or detecting a motion or presence of a person towards the door 200. In an embodiment, the dispenser 100 is adapted to spray the disinfectant on the door handle 202 after an elapse of a predetermined time after actuation of the door handle 202. In an embodiment, the dispenser 100 is actuated in response to an opening and closing of the door 200.

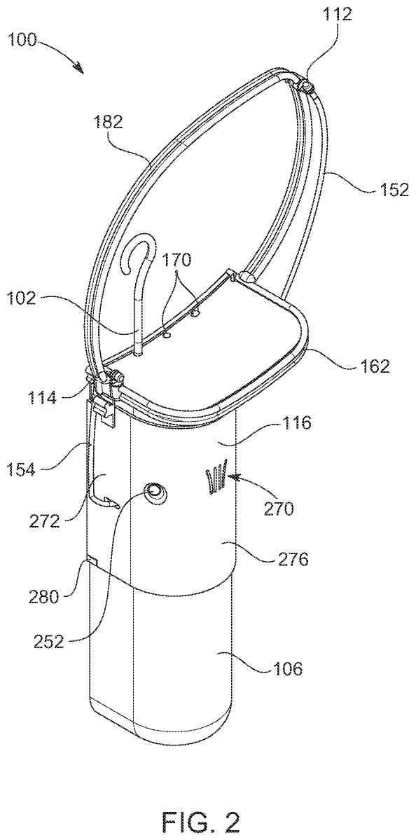

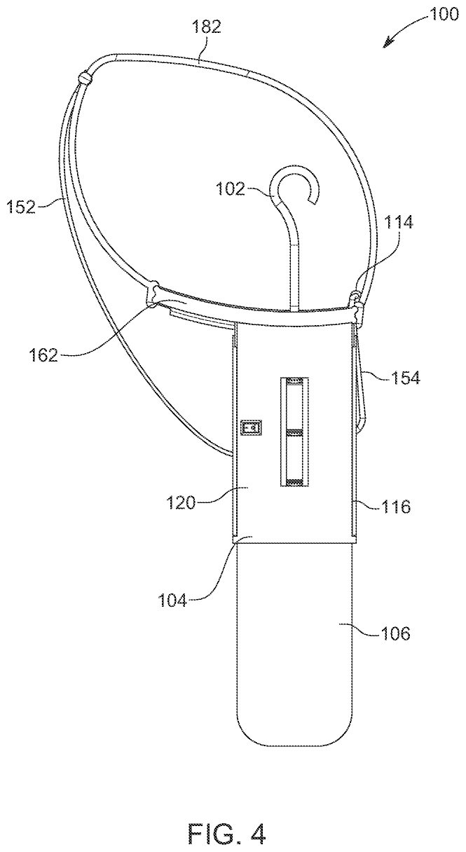

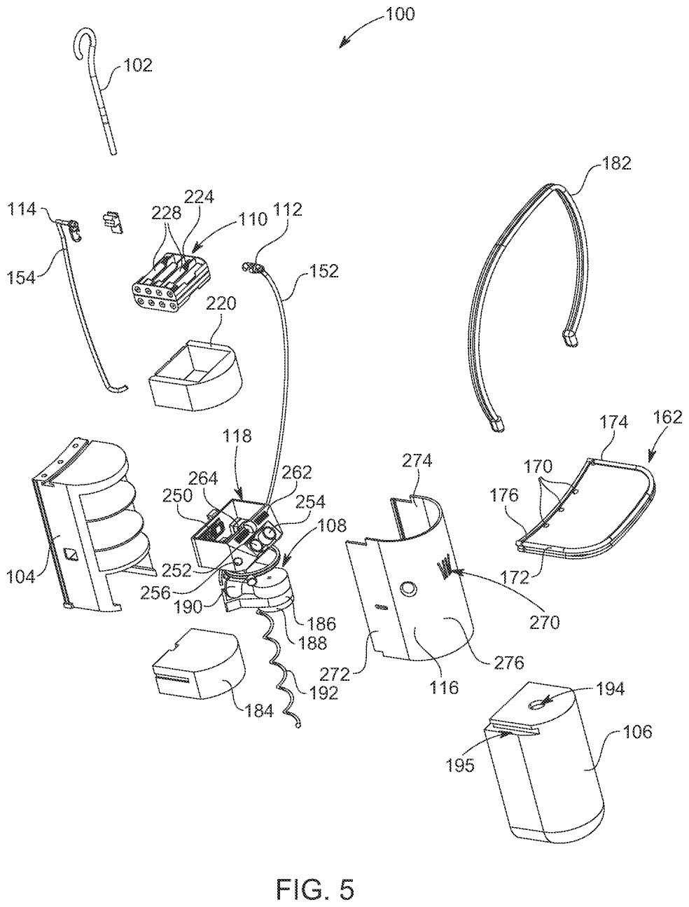

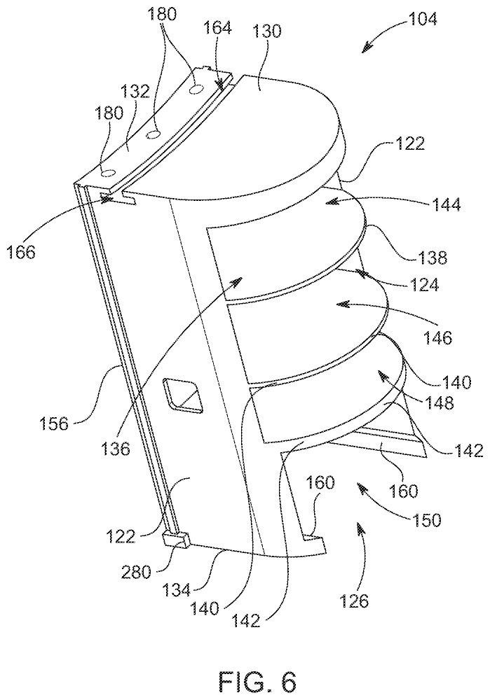

Now referring to FIGS. 2 to 9, the dispenser 100 includes a frame 104 (best shown in FIGS. 5, 6, 7, 10, and 11) adapted to support various components of the dispenser 100 and the hook 102 coupled to the frame 104 and adapted to mount the dispenser 100 on the door handle 202. As shown in FIGS. 5, 8, 9, the dispenser 100 also includes a container 106 for storing the disinfectant and coupled to the frame 104, an actuator assembly 108 to pump the disinfectant stored inside the container 106 to an ambient, a power storage unit 110 for providing electrical power to the actuator assembly 108, and at least one nozzle, for example, a first nozzle 112 and a second nozzle 114, fluidly connected to the actuator assembly, a cover 116 adapted to engage with the frame 104, and a control unit 118 to control the actuation of the actuator assembly 108. As shown in FIG. 6, the frame 104 includes a rear wall 120 (shown in FIG. 4) and a pair of sidewalls 122 extending from outwardly and substantially perpendicularly from the rear wall 120 and defining a first opening 124 and a second opening 126 therebetween. Further, the frame 104 includes a roof 130 attached to the rear wall 120 and the sidewalls 122 and arranged at a first longitudinal end 132 (i.e., upper end 132) of the frame 104. Accordingly, the second opening 126 is arranged at a second longitudinal end 134 (i.e., bottom end 134) of the frame 104, and the first opening 124 extends from the first longitudinal end 132 to the second longitudinal end 134. As shown, the rear wall 120, the sidewalls 122, and the roof 130 defining a storage chamber 136 therebetween such that the first opening 124 is the front opening of the chamber 136, while the second opening 126 defines the bottom opening of the chamber 136.

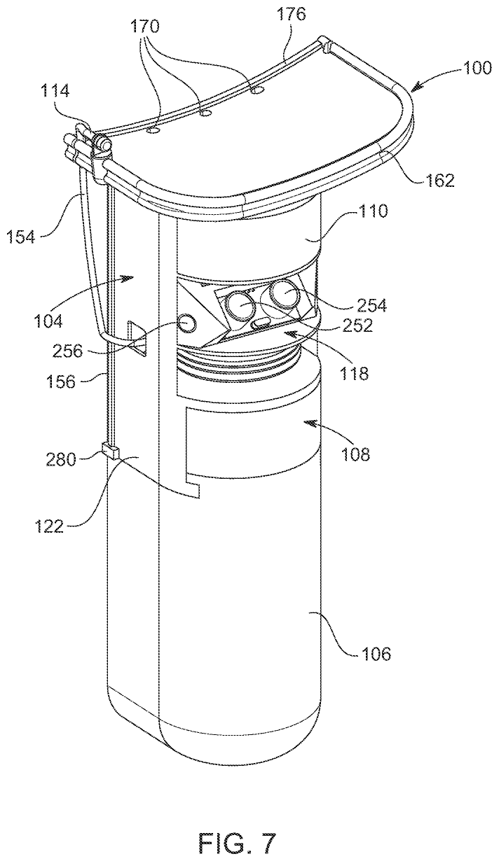

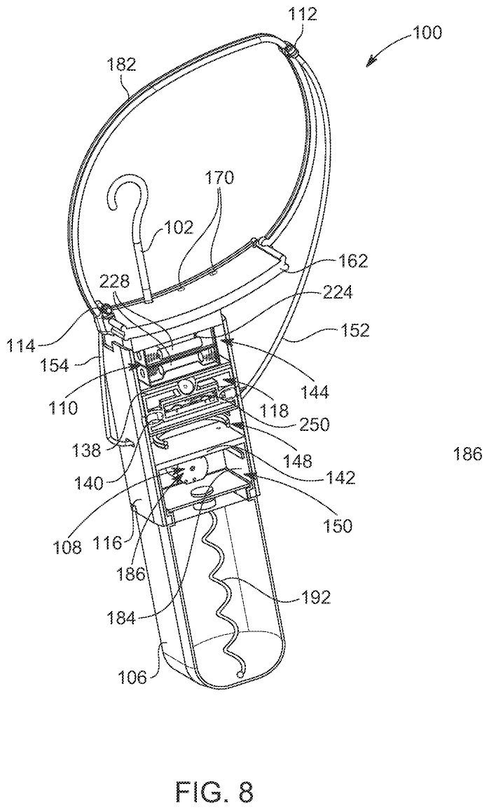

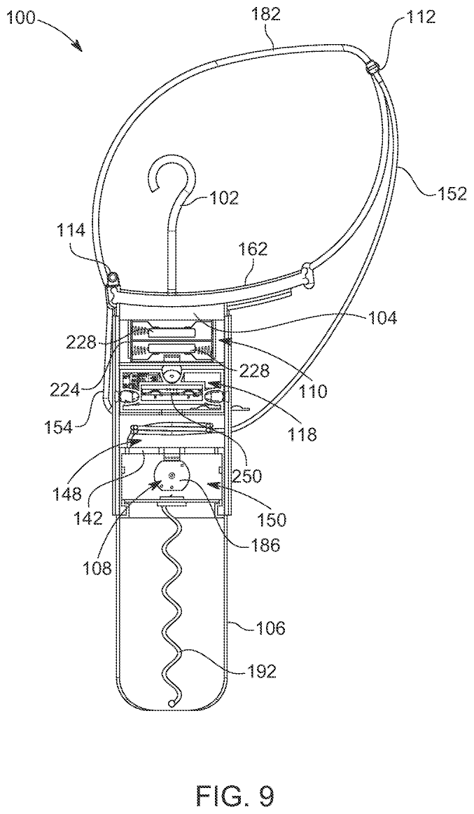

Additionally, the frame 104 includes a plurality of shelves, for example, three shelves 138, 140, 142, arranged inside the chamber 136 and arrayed in a vertical direction between the second longitudinal end 134 and the roof 130. The plurality of shelves divides the chamber into a plurality of compartments, for example, the three shelves 138, 140 142 divide the chamber 136 into four compartments 144, 146, 148, 150. As shown, each of the shelves 138, 140, 142 extends substantially parallel to the roof 130 and a first compartment 144 is defined between the roof 130 and a first shelf 138. The first compartment 144 is adapted to receive and support the power storage unit 110, as shown in FIGS. 8 and 9. A second compartment 146 is defined between the first shelf 138 and a second shelf 140 and is adapted to receive and support the control unit 118, as shown in FIGS. 7, 8, and 9. A third compartment 148 is defined between the second shelf 140 and a third shelf 142 and is adapted to receive tubes 152, 154 extending from the nozzles 112, 114 and store the extra lengths of the tubes 152, 154 (as shown in FIGS. 8 and 9, while a fourth compartment 150 defined between the third shelf 142 and the bottom opening 126 is adapted to receive and support the actuator assembly 108 and a portion of the container 106 (shown in FIGS. 8 and 9).

Moreover, the frame 104 includes a pair of first rails 156 extending from the first longitudinal end 132 to the second longitudinal end 134. The first rails 156 extend outwardly from the pair of sidewalls 122 and are arranged adjacent to and parallel with the rear wall 120. In embodiment, the first rails 156 constitute outwardly extensions of the rear wall 120. Also, the frame 104 includes a pair of second rails 160 arranged at the second longitudinal end 134 of the frame 104 and extending inwardly from the sidewalls 122 of the frame 104. Accordingly, each of the second rails 160 extend inside the fourth compartment 150 from the respective one of the sidewalls 122. To facilitate an attachment of a tray 162 to the frame 104, the roof 130 defines an engagement structure 164, for example, a groove 166 extending between the pair of sidewalls 122 and substantially parallel to the rear wall 120. As shown, the groove 166 is an inverted T-shaped groove to facilitate a sliding of a retention structure of the tray 162 within the groove 166 and relative to the roof 130 to enable a correct/desired positioning of the tray 162 relative to the roof.

As shown, the tray 162 includes an arcuate shape and is arranged abutting the roof 130 of the frame 104 and is supported by the frame 104. The tray 162, due its arcuate shape facilitates a collection of the dripping disinfectant. Also, as shown in FIGS. 1, 2, 7, and 8, portions of the tray 162 extends outwardly of the sidewalls 122 and the front opening 124 when engaged with the roof 130. Further, the tray 162 includes a plurality of holes 170 arrayed between a first side 172 and a second side 174 arranged opposite to the first side 174 of the tray 162. The plurality of holes 170 extends through the tray 162 and are arranged proximate to a rear end 176 of the tray 162. Each hole 170 facilitates an extension of the hook 102 through the tray 162 and inside an aligned hole 180 of the frame 104 and enables an attachment of the hook 102 with the frame 104 (i.e., the rear wall 120). In an embodiment, the holes 180 may be threaded hole to enable a threaded engagement of the hook 102 with the frame 104. The hook 102 is adapted to rotate about its central axis when attached to the frame 104 and the tray 162. In an embodiment, the hook 102 may rotate 360 degrees about the central axis. Further, the hook 102 is adapted to move linearly along the central axis. The linear and rotation movement of the hook 102 facilitates the mounting of the dispenser 100 (i.e., the nozzles 112, 114) in correct/desired orientation and position.

Further, the frame 104 includes an arch shaped handle 182 removably attached to the tray 162 and adapted to support the first nozzle 112. The handle 182 may be attached to the tray 162 by using snap on clips. Further, the first nozzle 112 may be removably attached to the handle 182 via a snap on clip. It may be appreciated that a position of the first nozzle 112 on the handle 182 may be adjusted so as to control a direction of the spray of the disinfectant. In an embodiment, the first nozzle 112 is attached to the handle 182 such that the spray is directed towards the door handle 202. Further, as shown, the second nozzle 114 is attached to the tray 162. As with the first nozzle 112, the second nozzle 114 is removable attached to the tray 162 via a snap on clip, and a position and an orientation of the second nozzle 114 may be adjusted to direct the spray in a desired direction. Although the first nozzle 112 and the second nozzle 114 are respectively shown to be engaged/attached with the handle 182 and the tray 162, it may be appreciated that both nozzles 112, 114 may either be connected to the handle 182 or the tray 162.

To receive the dispensing fluid from the container 106, each of the nozzles 112, 114 are connected to the actuator assembly 108 via the tubes 152, 154. The tubes 152, 154 extend inside the third compartment 148 through the openings into the cover 116 and aligned openings of the sidewalls 122 and then enter inside the fourth compartment 150 through the openings into the third shelf 142 and are connected to the actuator assembly 108. Extra lengths of the tubes 152, 154 may be stored inside the third compartment 148 to enable variable positioning of the nozzles 112, 114.

The actuator assembly 108, arranged inside the fourth compartment 150, is supported by the rear wall 120 and the sidewalls 122 of the frame 104. As shown, the actuator assembly 108 is arranged proximate to the third shelf 142 relative to the bottom opening 126, and includes a casing 184, and an actuator unit 186 arranged inside the casing 184. In an embodiment, the actuator unit 186 includes a pump 188 and a motor 190 to drive the pump 188 and facilitate a pumping of the disinfectant from the container 106. In such a case, the container 106 stores the disinfectant at a normal atmospheric pressure. The disinfectant stored inside the container 106 flows to the pump 188 via an inlet conduit 192 extending from the pump 188 to an inside of the container 106. The inlet conduit 192 may extend inside the container 106 through an opening 194 defined by a top wall of the container 106. Further, to facilitate an engagement and retention of the container 106 with the frame 104, the container 106 may define a pair of elongated grooves 195 arranged at a top end of the container 106 and adapted to receive and engage with the pair of second rails 160.

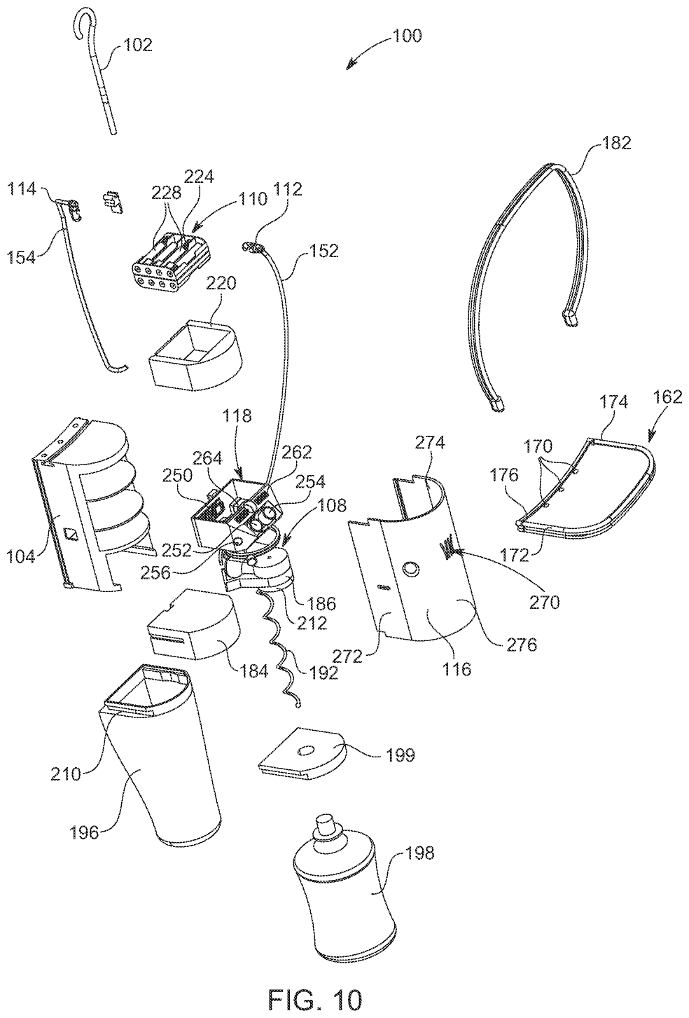

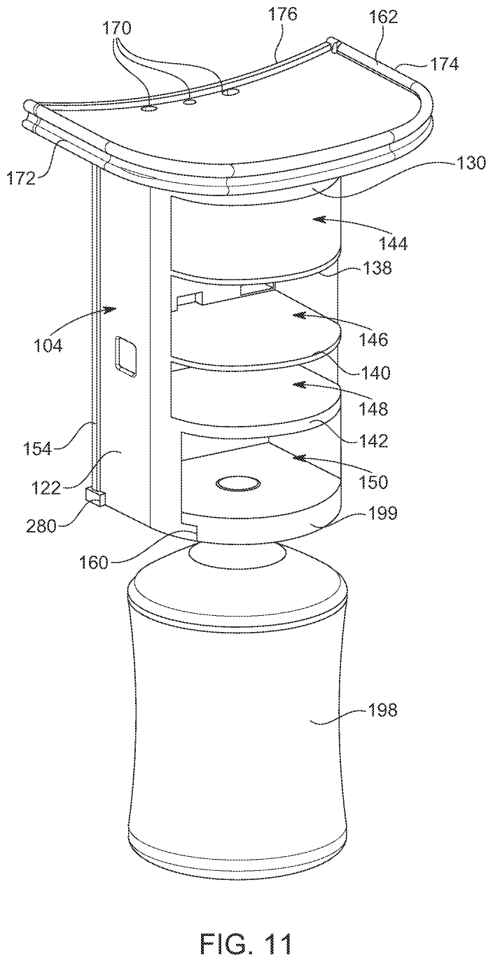

In an alternative embodiment, as shown in FIG. 10, the container 106 may be replaced with a container holder 196 and a bottle 198 adapted to arranged inside the container holder 196. The bottle stores the disinfectant at a predetermined pressure. As with the container 106, the container holder 196 also includes a pair of grooves 210 arranged spaced apart and substantially parallel to each other and disposed at an upper end of the holder 196 to receive the pair of second rails 160. Accordingly, the container holder 196 is engaged with the frame 104. Additionally, or optionally, the dispenser 100 may include an adapter plate 199 engaged with the bottle 198 to facilitate a connection/engagement of the bottle 198 with the frame 104. Further, in an assembly (as shown in FIG. 11), the adapter plate 199 is arranged inside the fourth compartment 150 and is engaged with the second rails 160. In such a case, the holder 196 may be omitted.

In such an alternative embodiment, the actuator unit 186 includes a valve 212 (shown in FIG. 10) connected to the pressurized bottle 198 and is adapted to provide the disinfectant to the nozzles 112, 114 when the valve 212 is actuated. In an embodiment, the valve 212 may be cam actuated valve such that the cam is actuated by a motor of the actuator unit 186 to facilitate a flow of the pressurized disinfectant to the nozzles 112, 114 via the tubes 152, 154 from the pressurized bottle 198. In an embodiment, the valve 212 may be a solenoid actuated valve.

Further, as shown in FIGS. 8 and 9, the actuator unit 186 is powered by the power storage unit 110 arranged in the first compartment 144. As best shown in FIGS. 5 and 10, the power storage unit 110 includes a housing 220 and a battery module 224 having at least one battery 228. In an embodiment, the at least one battery 228 may be a rechargeable battery. Alternately, the at least one battery 228 may be a non-rechargeable battery and is replaced after the end of the useful life of the battery 228. In some embodiments, the at least one battery 228 may be recharged by connecting the battery 228 with a power source via a suitable cable.

To control the supply of electricity to the actuator unit 186 and/or control the actuation of actuator assembly 108 (i.e., the pump 188 or the valve 212) to dispense disinfectant from the nozzles 112, 114, the control unit 118 includes a processor 250 (best shown in FIGS. 5, 8, 9 and 10), for example, a microcontroller. The at processor 250 may be embodied in several different ways. For example, the processor 250 may be embodied as one or more of various hardware processing modules such as a coprocessor, a microprocessor, a controller, a digital signal processor (DSP), a processing element with or without an accompanying DSP, or various other processing circuitry including integrated circuits such as, for example, an ASIC, an FPGA, a microcontroller unit (MCU), a hardware accelerator, a special-purpose computer chip, or the like. For example, the processor 250 may be a computer chip that includes a microcontroller with SPI (Serial Peripheral Interface), UART (Universal Asynchronous Receiver/Transmitter) and any other relevant interfaces that are known in the art. The computer chip may further include a clock, a crypto authenticator for network key security, one or more IoT devices such as Bluetooth, Wi-Fi, and SPI. Furthermore, the computer chip may include a push button interface, one or more user interface LEDs (Light Emitting Diode), or the like. In an example embodiment, the processor 250 may be operatively coupled with a memory storing a disinfect module. The memory may be embodied as SD (Secure Digital), RAM (Random Access Memory), ROM (Read Only Memory), hard-disk, or any combination thereof. The disinfect module may be a set of instructions (e.g., a software code), executed by the processor 250. The processor 250 may be configured to perform algorithms and operations described herein. Although the processor 250 is contemplated as the microcontroller, it may be appreciated that the processor may be a processing circuitry, ASIC circuitry, or any other electronic circuitry adapted to control various components, for example, the actuator unit 186, of the dispenser 100. Preferably, the processor 250 controls the actuation of the actuator unit 186 based on the inputs from at least one sensor of the control unit 118. In an embodiment, as best shown in FIGS. 5 and 10, the at least one sensor may include, at least one proximity sensor 252, at least one motion sensor 254, at least one position sensor 256, etc. The at least one proximity sensor 252 may be adapted to detect a presence of the person or the object near the dispenser 100. The at least one motion sensor 254 may be adapted to detect a motion of the person approaching towards or moving away from the dispenser 100. In some embodiments, the at least one motion sensor 254 may detect a motion/movement of the door handle 202 or the door 200. In an implementation, the at least one motion sensor 254 may be a time of flight sensor to detect a motion, a direction of motion, and a distance of the motion of an object or a person within a vicinity of the dispenser 100. In some scenarios, the motion sensor may be an accelerometer to detect an actuation of the door handle 202.

Based on the inputs from the at least one sensor 252, 254, 265, the processor 250 is configured to determine a movement of the door handle 202, a movement of a direction of the door 200, for example, an opening or a closing of the door 200, a presence of an object or a person near the door 200, or any combination thereof. Also, the processor 250 may determine a direction of the motion of the person relative to the dispenser 100, i.e., the processor 250 may determine if the person is moving towards or away from the dispenser 100. In response to the actuation of the door handle 202 and/or the detection of the person within a predefined distance from the dispenser 100 and direction of the motion of the person, the processor 250 actuates the actuator unit 186 (i.e., the pump 188 or the valve 212) and thereby causes an ejection/spray of the disinfectant on the door handle 202 and/or the door 200. In a further embodiment, the processor may also/alternatively cause the ejection/spray of the disinfectant on the hands of a person present in the vicinity of the dispenser 100. In an embodiment, the processor 250 may actuate the pump 188 or the valve 21 to spray the disinfectant upon an elapse of a predetermined time after detection of the movement of the door handle 202, the movement of the door 200, the detection of presence of the person within the predefined zone around the dispenser 100, or a combination thereof. The processor 250 may keep the actuator unit 186, i.e., the pump 188 or the valve 212 actuated for a predefined duration and deactivate the actuator unit 186 thereafter. For so doing, the processor 250 may control a closing and an opening of an electrical switch arranged between the battery module 224 and the actuator unit 186 to control the supply of power to the actuator unit 186. Although an automatic activation and deactivation of the actuator unit 186 based on the inputs from the at least one sensor is contemplated, it may be appreciated that the control unit 118 may include a switch, for example, a push button, in communication with the processor to enable a manual activation and deactivation of the actuation unit 186. Further, the processor 250 may include a timer to determine a time elapsed since a last/previous activation of the actuator unit 186. The processor 250 may be configured to activate the actuator unit 186 if the time elapsed since the previous activation has reached a predefined duration. In an embodiment, the predefined duration may be adjusted by a user, and exemplary predefined durations include one hour, two hours, four hours, and twenty four hours, for example.

In an embodiment, the control unit 118 may include at least one light source, for example, a first light source 262 and a second light source 264. The first light source may be an LED light and may indicate an activation or deactivation of the dispenser 100. In an embodiment, the first light source 262 may emit a green light when the dispenser 100 is activated, while the first light source 262 may emit a red light when the dispenser 100 is deactivated, and may flash red light when the dispenser is about to, currently, and/or has just sprayed disinfectant on the handle, for example 1 second before spraying, during spraying, and 3-5 seconds after spraying. Similarly, the second light 264 source may be an LED light and a color of the light emitted by the second light source 264 may indicate a charge status of the battery module 224. For example, a green light emitted by the second light source 264 may indicate that the battery module 224 has sufficient power, a yellow light emitted by the second light source 264 may indicate that the charge in the batteries 228 has decreased a predetermined charge, while a red light indicates a complete depletion of the charge of the batteries 228. To enable a view of the light emitted by the light sources 262, 264, the cover 116 includes a plurality of cut-outs 270 or transparent windows. Additionally, the dispenser 100 is adapted to generate one or more audio or visual signals to indicate that the door handle 202 has been recently sprayed, that the door handle 202 is about to be sprayed, and that the door handle 202 is again safe to be touched. In further embodiments, the dispenser may be powered by wired electric current in place of or in addition to the battery module 224.

Referring to FIG. 10, the cover 116 includes a pair of sidewalls 272, 274 arranged substantially parallel and spaced apart from each other, and a front wall 276 arranged between the pair of sidewalls 272, 274 and connecting the sidewalls 272, 274. In the illustrated embodiment, the front wall 276 is an arcuate wall with the pair sidewalls 272, 274 being arranged substantially tangentially to the front wall 276. The cover 116 is adapted to engaged with the frame 104 and covers the front opening 124 of the frame 104. As shown, in an assembly, ends of the sidewalls 272, 274 of the cover 116 are engaged with the pair of first rails 156. Further, the cover 116 is adapted to slide in a vertical direction along the first rails 156. To restrict or prevent the sliding of the cover 116 in the downward direction i.e., towards a base on the container 106, the frame 104 may include a pair of stoppers 280 arranged at the second longitudinal end 134 and below the pair of first rails 156. Further, in an assembly, an upper end of the cover 116 is arranged at a vertical offset from the tray 162 to facilitate a sliding movement of the cover 116 in a vertically upward direction to facilitate a removal or a replacement of the container 106 without disassembling the tray 162. Further, the cover 116 may be removed or disengaged by removing the tray 162 and the handle 182 from the frame 104.

An operation of the dispenser 100 is now explained. For sanitizing the door handle 202, or a person entering through the door 200, the dispenser 100 may be mounted in proximity to the door 200 or on the door 200, for example, on the door handle 202. For so doing, the hook 102 is rotated and/or linearly moved relative to its central axis to mount the dispenser 100 at the desired position and the desired orientation. Additionally, the nozzles 112, 114 may be adjusted to achieve the spray of the disinfectant from the nozzles 112, 114 in the desired directions. For example, the first nozzle 112 is arranged so that the disinfectant spray exiting the first nozzle 112 is directed towards the door handle 202 from a first direction, while the second nozzle 114 is arranged so as to direct the disinfectant spray towards the door handle 202 from a second direction, where the two sprays substantially cover the exposed surface of the door handle that a person opening the door 200 or operating the door handle 202 would likely touch. In an embodiment, the nozzles 112, 114 are arranged such the sprays from the nozzles 112, 114 are directed in the opposite direction. After suitably mounting the dispenser 100, a user may activate the dispenser 100 for operation. In an embodiment, the user may activate the dispenser 100 by operating a switch to an ON position. The activation of the dispenser 100 refers to a condition in which the processor 250 is able to activate or deactivate the actuator unit 186 to initiate the spray of disinfectant from the nozzles 112, 114 and stop the spray of disinfectant from the nozzles 114 in response to detection of one or more parameters or conditions. For example, the processor 250 may actuate the actuator unit 186 upon detection of an actuation or pressing of the door handle 202. In an embodiment, the processor 250 activates the actuator unit 186 (i.e., the pump 188 or the valve 212) after the elapse of a predetermined time from the actuation or pressing of the door handle 202. In some embodiments, the proximity sensors 256 may determine that the door handle 202 has reached its default position after detection of the movement of the door handle 202, and the processor 250 activates the actuator unit 186 upon such determination. The delay in time allows for the person who opened the door 200 to have released the door handle 202 and moved away from the door 200, such that the door handle 202 is free to be cleaned again.

In some embodiments, the processor 250 may detect a motion of a person or object towards the door 200 based on the inputs from one or more of the sensors 252, 254, 256, and accordingly actives the actuator unit 186 to dispense the disinfectant from the nozzles 112, 114. In some implementations, the processor 250 may automatically activate the actuator unit 186 in response to the elapse of the predefined time from the previous activation of the actuator unit 186 irrespective of detection of the actuation of the door handle 202, the opening of the door 200, the presence of a person in the vicinity of the door 200, the movement of the person or the object towards the door 200 (i.e., the dispenser 100), or any combination thereof. Further, the processor 250 may deactivate the actuator unit 186 when a predefined time has elapsed after the initiation of the spray from the nozzles 112, 114. In an embodiment, the processor 250 may keep the actuator unit 186 activated until the door 200 is opened or the sensors detects the presence of the person or object in proximity to the dispenser 100. Although, the dispenser 100 configured to spray the disinfectant or the cleaning liquid is shown and contemplated, it may be envisioned that the dispenser 100 may be similarly configured to emit UV light or any other light rays suitable to disinfect or clean surfaces upon detection of the actuation of the door handle 202, the opening of the door 200, the presence of a person in the vicinity of the door 200 or the dispenser 100, the movement of the person or the object towards the door 200 or the dispenser 100, or any combination thereof. The UV light sources could be powered by the battery module 224 and controlled by the processor 250. For so doing, the nozzles 112, 114 may be either replaced with suitable equipment, for example, UV ray emitters, or UV light sources may used in addition to the disinfectant and be placed adjacent to nozzles 112, 114 for extra sanitization. In a further embodiment, a heating source and fan are used in addition or alternatively to one or both of the disinfectant and the UV lights to disinfect the handle 202. The heating source and fan are preferably powered by the battery module 224 and controlled by the processor 250. The fan may direct heated air directly up towards the handle, or it may direct air through on or more tubes that can be directed at the handles.

As dispenser 100 can be hooked to the door handle 202 using the hook 102 and a position of the tray 162 can be adjusted relative to the frame 104 to balance the weigh the of dispenser and properly hang the dispenser 100 on the door handle 202, the dispenser 100 does not require complex installation. Also, the dispenser 100 is suitable for dispensing the disinfectant in any desired direction and multiple direction, the surface, such as, door handle surface is completely disinfected. Also, the tray 162 collects any dripping disinfectant, preventing a wetting of the floor. The modular structure of the dispenser 100 allows a customization of the device so that it can properly operate at minimal costs during high door traffic. In further embodiments, multiple hooks 102 may be used to hang the dispenser on push bars and other such door 200 openers. In further embodiment, three, four, or five sprayers are used to better surround and apply disinfectant to the door handle. In other embodiments the hook may be shaped in non-circular shapes, have internal acute, right, and/or obtuse angles, have clamps, and other manners of vertically securing the frame to the handle. In further embodiments, the hook may be omitted and the dispenser may be attached directly to the door with Velcro, screws, glue or other such mechanical or chemical attachment.

The invention illustratively disclosed herein suitably may explicitly be practiced in the absence of any element which is not specifically disclosed herein. While various embodiments of the present invention have been described in detail, it is apparent that various modifications and alterations of those embodiments will occur to and be readily apparent those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. The present disclosure also contemplates other embodiments “comprising,” “consisting of” and “consisting essentially of,” the embodiments or elements presented herein, whether explicitly set forth or not. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items, while only the terms “consisting of” and “consisting only of” are to be construed in the limitative sense.

Wherefore, I/we claim:

1. An automatic door handle/panel sanitizer dispensing assembly comprising:
a frame;
a control unit including a processor;
an actuator assembly controlled by the processor;
a first sensor operatively connected to the processor;
a power storage unit powering the control unit, the actuator assembly, and the first sensor; and
a first nozzle fluidly connected to the actuator assembly.

2. The assembly of claim 1 further comprising a second nozzle fluidly connected to actuator assembly.

3. The assembly of claim 1 further comprising a container coupled to the frame, for storing disinfectant.

4. The assembly of claim 1 wherein the actuator assembly includes one of a pump and a valve.

5. The assembly of claim 1 further comprising a tray having an arcuate shape and being arranged abutting a roof of the frame and being supported by the frame.

6. The assembly of claim 5 further comprising a plurality of tray holes extending through the tray arranged proximate to a rear end of the tray.

7. The assembly of claim 6 further comprising a hook to hang the assembly onto the door handle and wherein each tray hole facilitates an extension of the hook through the tray and inside an aligned frame hole of the frame, and enables an attachment of the hook with the frame.

8. The assembly of claim 7 wherein the hook is threaded and the frame holes are threaded to enable a threaded engagement of the hook with the frame.

9. The assembly of claim 5 further comprising an arch shaped handle removably attached to the tray and adapted to support one of the first nozzle, a second nozzle, and both the first nozzle and the second nozzle.

10. The assembly of claim 1 wherein the first sensor is one of a proximity sensor, a motion sensor, and a position sensor.

11. The assembly of claim 1 wherein the control unit is programed such that when the first sensor detects an object is one of in proximity to the door handle and touches the door handle, and then the object is removed from the door handle, the processor causes the actuator assembly to spray disinfectant on the door handle.

12. The assembly of claim 1 wherein the control unit is programed such that when the first sensor detects the door is opened and then closed again, the processor causes the actuator assembly to spray disinfectant on the door handle.

13. The assembly of claim 1 further comprising a UV light source controlled by the control unit and powered by the power storage unit, the UV light source arranged to direct UV light rays onto the door handle when actuated by the control unit.

14. The assembly of claim 1 further comprising a heating element and a fan, both controlled by the control unit and powered by the power storage unit, the fan arranged to direct heated air onto the door handle when actuated by the control unit.

15. An automatic door handle/panel sanitizer dispensing assembly comprising:
a frame;
a control unit including a processor;
an actuator assembly controlled by the processor, the actuator assembly includes one of a pump and a valve;
a first sensor operatively connected to the processor;
a power storage unit powering the control unit, the actuator assembly, and the first sensor;
a first nozzle and a second nozzle, both fluidly connected to the actuator assembly;
a container coupled to the frame, for storing disinfectant;
a threaded hook to hang the assembly onto the door handle;
a tray having an arcuate shape and being arranged abutting a roof of the frame and being supported by the frame;
a plurality of threaded tray holes extending through the tray arranged proximate to a rear end of the tray, wherein each tray hole facilitates an extension of the hook through the tray and inside an aligned frame hole of the frame, and enables an attachment of the hook with the frame;
an arch shaped handle removably attached to the tray and adapted to support one of the first nozzle, a second nozzle, and both the first nozzle and the second nozzle; and
one of
a UV light source controlled by the control unit and powered by the power storage unit, the UV light source arranged to direct UV light rays onto the door handle when actuated by the control unit, and
a heating element and a fan, both controlled by the control unit and powered by the power storage unit, the fan arranged to direct heated air onto the door handle when actuated by the control unit;
wherein the first sensor is one of a proximity sensor, a motion sensor, and a position sensor, and
wherein the control unit is programed such that one of
when the first sensor detects an object is one of in proximity to the door handle and touches the door handle, and then the object is removed from the door handle, the processor causes the actuator assembly to spray disinfectant on the door handle; and
when the first sensor detects the door is opened and then closed again, the processor causes the actuator assembly to spray disinfectant on the door handle.
